(19) United States
(12) Patent Application Publication
(10) Pub. No.: US 2005/0021003 A1
(43) Pub. Date: Jan. 27, 2005

(54) Apparatus for directional guidance of a vascular device and method of use

(76) Inventors: Richard Caso, San Juan Capistrano, CA (US); R. Brand Caso, San Clemente, CA (US)

Correspondence Address: GENE SCOTT; PATENT LAW & VENTURE GROUP, 3140 RED HILL AVENUE, SUITE 150, COSTA MESA, CA, 92626-3440, US

(21) Appl. No.: 10/686,757

(22) Filed: Oct. 15, 2003

Related U.S. Application Data
(60) Provisional application No. 60/462,615, filed on Apr. 14, 2003.

(51) Int. Cl. A61M 25/01

(52) U.S. Cl. 604/528; 600/434

(57) ABSTRACT

An apparatus and method for directional guidance of a vascular device includes a flexible insertion device terminating in a distal end and a channel formed in the insertion device so as to intersect the distal end. The insertion device comprises an inner guiding catheter and an outer deflecting catheter configured with a tubular wall defining an interior lumen such that the deflecting catheter is positioned coaxially and slidably about the guiding catheter along at least a portion of the lumen, the tubular wall having the channel formed therein so as to communicate with the lumen and intersect the deflecting catheter's distal tip. In use, the distal tip of the deflecting catheter is seated within a body lumen and the guiding catheter is retracted so that a vascular device, approaching from a lateral direction, may then be slidably received within the exposed channel and guided into the body lumen.

### RELATED APPLICATIONS

This application claims priority and is entitled to the filing date of U.S. Provisional application Ser. No. 60/462,615 filed Apr. 14, 2003, and entitled “Apparatus for Directional Guidance of a Catheter.” The contents of the aforementioned application are incorporated by reference herein.

### BACKGROUND OF THE INVENTION

### INCORPORATION BY REFERENCE

Applicant(s) hereby incorporate herein by reference, any and all U.S. patents, U.S. patent applications, and other documents and printed matter cited or referred to in this application.

1. Field of the Invention

This invention relates generally to surgical methods, and more particularly to a catheter apparatus and method of use for directional guidance of vascular devices.

2. Description of Related Art

The following art defines the present state of this field:

Douglas, U.S. Pat. No. 4,195,624 describes a device for facilitating insertion of an endoscope into the esophagus which includes an elongated tube composed of a flexible elastomer, the tube having a substantially uniform outer diameter along most of its length and a solid tapered tip end, the tip end being joined to the remainder of the tube by means of a hollow tapered neck portion. The tube is provided with an opening behind the neck of generally elongated configuration. The geometry of the opening and the flexibility of the tube are such that when the distal end of an endoscope is inserted into the aperture, the distal end is slightly bent and is received in wedged engagement within the tapered neck portion, and a portion immediately behind the distal end of the endoscope is resiliently supported by a portion of the periphery of the opening formed in the tube.

Arenas et al., U.S. Pat. No. 4,676,249 describes a multi-mode guidewire selectively allowing the creation of varying degrees of flexibility at varying locations of the guidewire. An elongate coiled wire body of the guidewire is capable of assuming an arcuate shape adjacent to its capped distal end. The coiled wire body may be made less flexible by a curve control core wire which is positionable by means of a knob at its proximal end. The curve control core wire includes a region of moderate flexibility in its distal region which can be stiffened by a stiffening member. The stiffening member is positionable by a handle located at its proximal end. A method of advancing a catheter and the guidewire includes selecting a most advantageous mode of use of the guidewire for advancement.

Hawkins et al., U.S. Pat. No. 4,799,495 describes a localization needle assembly including an outer tubular cannula and an inner needle slidably mounted for movement within the outer cannula between extended and retracted portions, the needle having a pointed tip which projects from the front end of the assembly when the needle is extended while the surgeon locates a lesion.

When the tip is retracted, a barb, which is secured to the needle, is deployed through an opening in the sidewall of the outer cannula when the needle is retracted, the barb anchoring the needle assembly in body tissue in the proximity of the lesion. Detachable handles are provided for locking the inner needle and outer cannula together and to facilitate extension and retraction of the needle. In one embodiment, the outer cannula has a helical screw tip for securing the needle assembly to a body organ or body tissue. A guide assembly is advanced along the needle assembly, which has been inserted into the body tissue to locate a lesion to position one end of a guide wire at the lesion to facilitate the introduction of a surgical instrument into the body and guidance of same directly to the lesion.

Jacobsen et al., U.S. Pat. No. 5,916,194 describes a catheter/guide wire steering mechanism including a catheter having a proximal end, a distal end, and sidewalls which define at least the first lumen. An opening is formed in a sidewall of the catheter, near the distal end thereof in communication with the first lumen. A plug is disposed in the catheter at the distal end thereof and includes a curved surface for deflecting and directing out the opening, the leading end of a guide wire (or other catheter) inserted into the lumen at the proximal end of the catheter. This enables guiding the guide wire laterally from the catheter either into a passageway branching from the main passageway into which the catheter is inserted, or to perforate a sidewall of the main passageway.

Haaga, U.S. Pat. No. 6,162,203 describes a cargo delivery device having coaxial, telescopically interengaged cargo delivery needle, an outer cannulas which are axially and rotatably displaceable relative to one another. The cargo delivery needle has a distal portion provided with a cargo recess for carrying a cargo to a site in a patient.

Burney et al., U.S. Pat. No. 6,203,524 describes a guide for biopsy and microtherapy which includes an introducer cannula defining a lumen sized to receive a diagnostic or therapeutic item therethrough and a lateral opening in communication with the lumen adjacent the first end of the cannula. The invention also includes a solid tip having an anatomically distal end secured to the first end of the cannula and a proximal end configured to pierce tissue. A ramp is disposed within the cannula at an end of the lateral opening adjacent the first end of the cannula. The ramp is inclined toward the lateral opening, whereby the item will be deflected through the lateral opening as it advances within the lumen and exits the cannula. In some embodiments, the item is a biopsy needle, ablation means or a radiopharmaceutical seed. The invention also includes methods of obtaining a biopsy sample and methods for treating lesions.

Burney et al., U.S. Pat. No. 6,447,477 describes a guide for biopsy and microtherapy which includes an introducer cannula defining a lumen sized to receive a diagnostic or therapeutic item therethrough and a lateral opening in communication with the lumen adjacent the first end of the cannula. The invention also includes a solid tip having an anatomically distal end secured to the first end of the cannula and a proximal end configured to pierce tissue. A ramp is disposed within the cannula at an end of the lateral opening adjacent the first end of the cannula. The ramp is inclined toward the lateral opening, whereby the item will be deflected through the lateral opening as it advances within the lumen and exits the cannula. In some embodiments, the item is a biopsy needle, ablation means or a radiopharmaceutical seed. The invention also includes methods of obtaining a biopsy sample and methods for treating lesions.

Carrillo, Jr. et al., U.S. Pat. No. 6,52,951 describes a single operator exchange biliary catheter having a common distal lumen. The biliary catheter includes an elongate shaft having a proximal portion defining an ancillary lumen and a distal portion defining a common guidewire and ancillary lumen. The common distal lumen reduces the profile of the distal portion of the shaft. The elongate shaft also includes a proximal guidewire port disposed between the proximal end of the shaft and the distal end of the shaft to facilitate single operator use. A seal may be disposed adjacent the proximal guidewire port to thereby seal the port. Preferably, the shaft includes a single lumen distal portion and a bitumen proximal portion. The single lumen distal portion of the shaft may be curved and may include a tapered or spherically shaped distal tip.

Mehier, U.S. 2001/0034502 describes a device for directly delivering an active substance within all or part of a human or animal tissue cell, characterized in that it is in the form of a hollow tube (1), whereof the walls in contact with said tissue are provided with perforations (5) and whereof the distal end (2) is sealed, while the proximal end (3) is shaped so as to receive removable closing means, said tube being capable of bearing a pressure of at least 50 bars.

Our prior art search with abstracts described above teaches a tubular sheath for facilitating the insertion of an endoscope, a multi-mode guidewire, a localization needle assembly, a catheter/guide wire steering apparatus and method, a cargo delivery needle, a surgical and pharmaceutical site access guide and methods, a rapid exchange catheter with detachable hood, and a device for directly delivering an active substance within a cell tissue and means for implanting said device and appliances for injecting active substance into said device, but does not teach a catheter apparatus configured with an open-ended channel so as to guide or direct a non-coaxially introduced vascular device such as a catheter, guidewire, balloon catheter or pacing electrode catheter. Specifically, in the context of the difficult task of placing such a vascular device in the coronary sinus when the device has entered the heart's right atrium through the superior vena cava, the prior art does not teach an apparatus and method that when placed in the right atrium through the inferior vena cava, has the capability to guide a vascular device into the coronary sinus. The present invention fulfills these needs and provides further related advantages as described in the following summary.

### SUMMARY OF THE INVENTION

The present invention teaches certain benefits in construction and use which give rise to the objectives described below.

The present invention is an apparatus and method of use for directional guidance of a vascular device and includes a flexible insertion device terminating in a distal end with a channel formed in the insertion device so as to intersect the distal end. The insertion device comprises an inner guiding catheter and an outer deflecting catheter configured with a tubular wall defining an interior lumen such that the deflecting catheter is positioned coaxially and slidably about the guiding catheter along at least a portion of the lumen, the tubular wall having the channel formed therein so as to communicate with the lumen and intersect the deflecting catheter's distal tip. The deflecting catheter may be further configured with a pre-formed bend.

In use, the apparatus is advanced along a first body lumen and into a body cavity where it is then manipulated so as to seat the guiding catheter's distal tip within a second body lumen. The pre-formed bend of the deflecting catheter facilitates orienting the guiding catheter's distal tip adjacent to the second body lumen. The apparatus is then advanced within the second body lumen such that the channel formed in the deflecting catheter is partially positioned within the second body lumen and partially positioned within the body cavity. With the apparatus so positioned, the guiding catheter is retracted and the vascular device is advanced along a third body lumen and into the body cavity. The vascular device's distal tip is then positioned within the exposed channel, enabling the vascular device to be advanced into the second body lumen as guided by the channel. Retracting the apparatus within the first body lumen leaves the distal tip of the vascular device positioned within the second body lumen.

A primary objective of the present invention is to provide an apparatus and method of use of such apparatus that provides advantages not taught by the prior art.

Another objective is to provide such an invention capable of directionally guiding a non-coaxially introduced vascular device.

A further objective is to provide such an invention capable of being retracted after directionally guiding a non-coaxially introduced vascular device.

A still further objective is to provide such an invention capable of being advanced through a first body lumen and into a body cavity to be partially seated within a second body lumen so that a vascular device entering the body cavity through a third body lumen may be guided into the second body lumen.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the present invention. In such drawings:

FIG. 1 is a perspective view of the preferred embodiment of the invention shown in its pre-formed, bent configuration with a fluid dispensing device adjacent its proximal end;

FIG. 2 is a partial top view of a deflecting catheter thereof; and

FIGS. 3-8 are schematic diagrams showing the sequence of the preferred method of use thereof for guiding a vascular device within the heart's right atrium.

### DETAILED DESCRIPTION OF THE INVENTION

The above described drawing figures illustrate the invention in at least one of its preferred embodiments, which is further defined in detail in the following description.

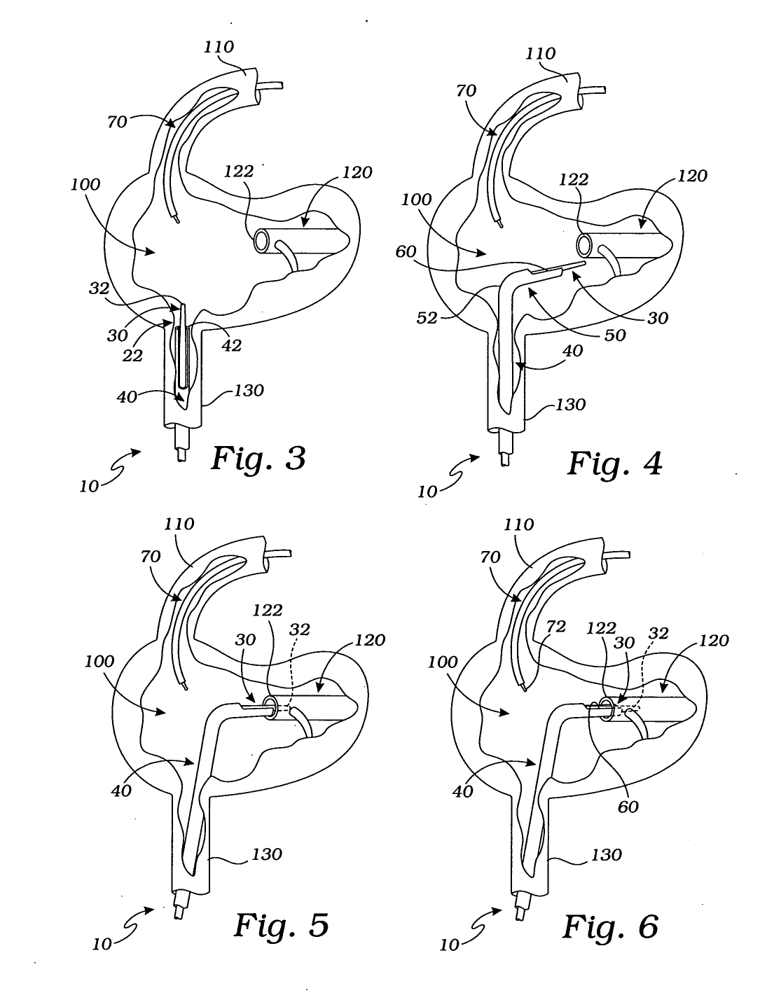

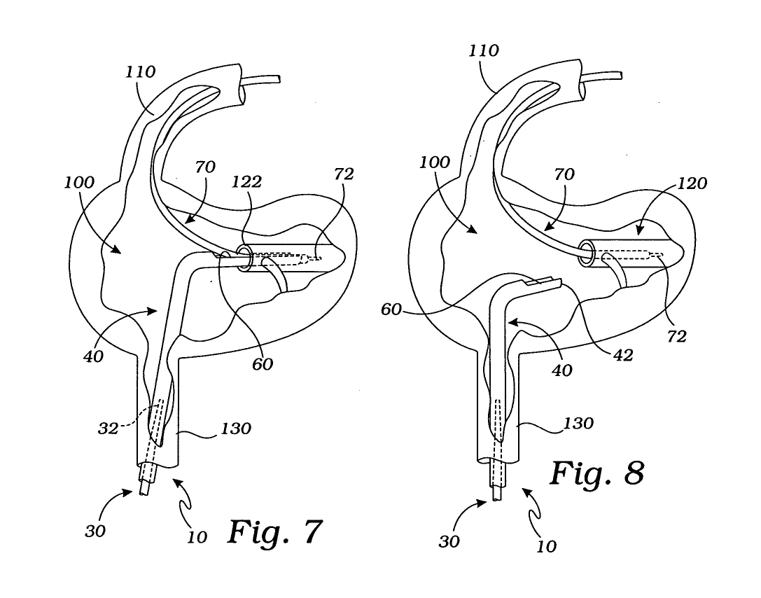

The present invention is an apparatus 10 for directional guidance of a vascular device 70 (FIGS. 3-8) generally comprising a flexible insertion device 20 terminating at a distal end 22 and a channel 60 (FIG. 2) formed in the insertion device 20 so as to intersect the distal end 22, the channel 60 being configured to slidably receive the vascular device 70 and guide the vascular device 70 toward the distal end 22. In a preferred embodiment, the insertion device 20 comprises an inner guiding catheter 30 and an outer deflecting catheter 40. The deflecting catheter 40 terminates at a distal deflecting tip 42 and is configured with a tubular wall 44 defining an interior lumen 46 (FIG. 2) such that the deflecting catheter 40 is positioned coaxially and slidably about the guiding catheter 30 along at least a portion of the lumen 46. The guiding catheter 30 is formed with a tapered distal guiding tip 32. As best shown in FIG. 2, the channel 60 is formed in the tubular wall 44 of the deflecting catheter 40 so as to communicate with the lumen 46 and intersect the deflecting tip 42. While the channel 60 is shown as having a substantially rectangular profile, it will be appreciated that other profiles, such as elliptical and semi-elliptical, may be employed as well. The channel 60 is formed having a sufficient length, width and depth so as to transversely receive and axially guide the vascular device 70, as explained more fully below. In the preferred embodiment, the channel is approximately 1.5″ long, approximately 0.1″ wide, and approximately 0.07″ deep, though it will be appreciated by those skilled in the art that the channel can take on different dimensions to optimally suit different applications. The deflecting catheter 40 has a relatively long, substantially linear proximal portion 48 and a relatively short, substantially linear distal portion 50. A pre-formed bend 52 of up to about 85 angular degrees is formed between the proximal and distal portions such that the distal portion 50 is angled relative to the proximal portion 48 so as to have a superior aspect 54 in which the channel 60 is formed. The guiding catheter 30 may be formed with a complimentary bend. Both catheters are formed through a conventional extrusion technique from a flexible, resilient, medical-grade plastic so as to have structural memory, as is known and used in the art, that is, the plastic material, once formed into a desired shape, retains memory of that shape and is thereafter biased to resume that shape after being deformed into an alternate shape. Such structural memory is achieved in the present invention by the cooperation of the plastic material and the pre-formed bend 52 and this enables the catheter apparatus 10 to function as described below. The deflecting tip 42 of the deflecting catheter 40 is formed with a corner-break 56 so as to produce a smooth transition with the guiding catheter 30 when the guiding catheter 30 is coaxially inserted within the interior lumen 46 of the deflecting catheter 40 such that the guiding catheter's guiding tip 32 protrudes beyond the deflecting catheter's deflecting tip 42. The opposite proximal end 58 of the deflecting catheter 40 is formed with a hemostatic valve as is known in the art so as to prevent fluids from passing along the lumen 46 and out the deflecting catheter's proximal end 58. The deflecting catheter 40 is preferably a size 10 or 11 French, though it will be appreciated by those skilled in the art that other catheter sizes may be employed without departing from the spirit and scope of the invention. The guiding catheter 30 is formed with an axial through-hole 34 communicating between the guiding catheter's proximal and distal ends for the passage of a guidewire (not shown) or the delivery of fluids therethrough. The proximal end 36 of the guiding catheter 30 is configured with a universal hub connection 38 to facilitate attaching a fluid delivery device 80, such as a syringe, fluid line connector or other device, to the guiding catheter 30. While the preferred embodiment of the present invention is shown and described as a pair of coaxially positioned catheters, it will be appreciated by those skilled in the art that the flexible insertion device 20 of the directional guidance apparatus 10 may be comprised of various other combinations of catheters, including a single catheter, without departing from the spirit and scope of the invention.

In a preferred use, the apparatus 10 of the present invention is employed to provide mechanical guidance of a vascular device 70, such as a catheter, guidewire, balloon catheter, or pacing electrode catheter, introduced through the superior vena cava 110 into the right atrium 100 and directed toward the os 122 of the coronary sinus 120. Such a procedure is often employed in the specific context of biventricular cardiac pacing, wherein one or two pacing electrodes (not shown) are placed in a heart's right ventricular chamber, or right atrium 100, and a third pacing electrode, or vascular device 70 is placed in a branch of the coronary sinus 120 (FIG. 8) to effect pacing of the left ventricle (not shown). As is known in the art, placement of pacing electrodes 70 in the coronary sinus 120 has been challenging due to the lack of visible landmarks, the variable shape, size and location of the os 122 of the coronary sinus 120, the variable anatomy of the branches (not shown) of the coronary sinus 120 beyond the os 122, and the pumping motion of the heart (not shown). While the catheter apparatus 10 of the present invention is particularly well-suited for guiding and directing such a pacing electrode 70 into the coronary sinus 120, as explained in more detail below, it will be appreciated that the apparatus 10 and its method of use are also capable of functioning to guide and direct catheters and the like in any medical procedure in which it is advantageous to provide the mechanical guidance from a direction or body lumen other than that of the catheter itself.

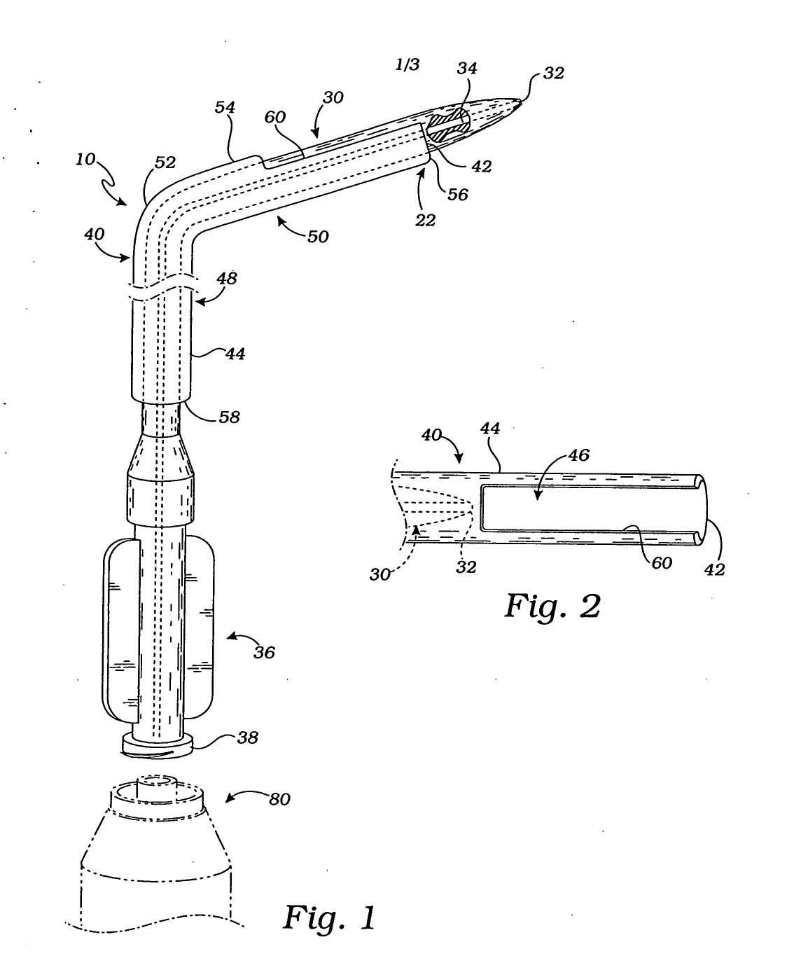

For the preferred embodiment and method of use, the catheter apparatus 10 of the present invention is configured as described above with the deflecting catheter 40 coaxially and slidably positioned about the guiding catheter 30 such that the guiding catheter's guiding tip 32 protrudes beyond the deflecting catheter's deflecting tip 42 so as to cooperate in forming a tapered distal end 22. As such, the apparatus 10 is configured substantially as shown in FIG. 1 and is ready for insertion within a body lumen. In the present exemplary application, the catheter apparatus 10 is advanced through either the right or left femoral vein (not shown) and into a first body lumen 130, the inferior vena cava. Because the guiding catheter 30 and deflecting catheter 40 are formed from a flexible plastic, it will be appreciated that the catheter apparatus 10 is able to flex from its pre-formed, bent configuration so as to conform to the anatomical structures through which the apparatus 10 is being advanced; in this case, the inferior vena cava 130. Thus, as best shown in FIG. 3, the catheter apparatus 10 is substantially linear as it passes through the inferior vena cava 130 and begins to enter the body cavity or right atrium 100. Upon further advancement of the catheter apparatus 10, as shown in FIG. 4, the distal portion 50 and the bend 52 of the outer deflecting catheter 40 is located within the right atrium 100 and the deflecting catheter 40 is then free to return to its original shape. The apparatus is then manipulated so as to orient the angled distal portion 50, and the distal tip 32 of the guiding catheter 30, specifically, toward the os 122 of the second body lumen, the coronary sinus 120. As shown, because the channel 60 is located on the superior aspect 54 of the deflecting catheter's distal portion 50, the channel 60 is oriented toward the third body lumen, the superior vena cava 110, through which the vascular device 70 is introduced. This orientation is critical to the guiding function of the catheter apparatus 10, as explained more fully below. With the catheter apparatus 10 so positioned within the right atrium 100, the apparatus 10 is then manipulated as shown in FIG. 5 so as to seat the tapered distal tip 32 within the os 122 of the coronary sinus 120. It will be appreciated by those skilled in the art that the tapered distal tip 32 serves to locate within the os 122 of the coronary sinus 120 while reducing the risk of damaging any of the vascular anatomy. Once this positioning of the catheter apparatus 10 is verified, as by fluoroscopic confirmation, the apparatus 10 is advanced further within the coronary sinus 120 such that the channel 60 is partially positioned within the coronary sinus 120 and partially positioned within the right atrium 100. As with the tapered distal tip 32 of the guiding catheter 30, the corner-break 56 (FIG. 1) formed on the distal tip 42 of the deflecting catheter 40 also reduces the risk of trauma to the os 122 and the coronary sinus 120 as the catheter apparatus 10 is advanced to the seated position shown in FIG. 6. With the catheter apparatus 10 so seated within the coronary sinus 120, the vascular device 70 is advanced within the superior vena cava 110 and into the right atrium as shown in FIGS. 3-6. As shown in FIG. 7, the guiding catheter 30 is then retracted within the deflecting catheter 40 so as to expose the interior lumen 46 (FIG. 2) to the right atrium 100 through the channel 60. Next, the vascular device 70 is advanced further into the right atrium 100 so as to locate its distal tip 72 within the channel 60. Because the channel 60 is formed in the superior aspect 54 of the deflecting catheter 40 such that the channel 60 is oriented generally toward the superior vena cava 110 when the deflecting catheter 40 is seated within the coronary sinus 120, it will be appreciated that the distal tip 72 of the vascular device 70 is then more readily located within the channel 60. Once the distal tip 72 is so located, the vascular device 70 is simply advanced into the coronary sinus 120 as guided by the channel 60. The deflecting catheter 40 may then be retracted about the guiding catheter 30 within the inferior vena cava 130 to leave the distal tip 72 of the vascular device 70 positioned within the coronary sinus 120. It will be appreciated by those skilled in the art that the formation of the channel 60 within the deflecting catheter 40 so as to intersect the distal tip 42, and thereby form a distally-open-ended channel, facilitates the retraction of the deflecting catheter 40 while leaving the vascular device 70 in position, when, as here, the vascular device 70 is introduced along a direction or body lumen other than that of the catheter apparatus 10.

While the invention has been described with reference to at least one preferred embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

1. An apparatus for directional guidance of a vascular device, comprising:
a generally linear insertion device having a medially positioned bend, the insertion device biased to the bend by structural memory of the plastic, the insertion device terminating at a distal end; and
an axially oriented channel formed in the insertion device so as to intersect the distal end, the channel being open to an outer surface of the insertion device and configured by a channel width of at least 0.1 inches, a channel depth of at least 0.07 inches, and a channel length of at least 1.5 inches, so as to slidably receive the vascular device and to guide the vascular device toward the distal end.

2. The apparatus of claim 1 wherein the insertion device comprises:
an inner guiding catheter; and
an outer deflecting catheter configured with a tubular wall defining an interior lumen such that the guiding catheter is positioned coaxially and slidably within the deflecting catheter along at least a portion of the lumen, the tubular wall having the channel formed therein so as to communicate with the lumen.

3. The apparatus of claim 2 wherein:
the deflecting catheter has a relatively long, substantially linear proximal portion and a relatively short, substantially linear distal portion, the deflecting catheter being further configured with the bend formed therein at an angle of up to about 85 angular degrees between the proximal and distal portions such that the distal portion is angled relative to the proximal portion so as to have a superior aspect; and
the channel is formed in the superior aspect.

4. The apparatus of claim 2 wherein the distal end terminates with a broken corner so as to produce a smooth transition with the guiding catheter.

5. The apparatus of claim 2 wherein the proximal end of the deflecting catheter is formed with a hemostatic valve, the valve functional for preventing fluids from exiting the lumen.

6. The apparatus of claim 2 wherein the deflecting catheter is one of a size 10 French and a size 11 French.

7. The apparatus of claim 2 wherein the guiding catheter terminates with a tapered guiding tip.

8. The apparatus of claim 2 wherein the guiding catheter is formed with an axial through-hole communicating between the proximal and distal ends of the guiding catheter enabling passage of a guidewire and the delivery of fluids therethrough.

9. The apparatus of claim 8 wherein the guiding catheter is configured on a proximal end with a universal hub having means for receiving a fluid delivery device attached to the guiding catheter.

10. An apparatus for directional guidance of a vascular device into a body lumen, comprising:
an inner guiding catheter; and
an outer deflecting catheter coaxially and slidably positioned about the guiding catheter, the deflecting catheter being configured with a means for slidably receiving the vascular device from a lateral direction and for axially guiding the vascular device into the body lumen.

11. An apparatus for directional guidance of a vascular device comprising:
an inner guiding catheter; and
an outer deflecting catheter configured with a tubular wall defining an interior lumen such that the deflecting catheter is positioned coaxially and slidably about the guiding catheter along at least a portion of the lumen, the tubular wall having a channel formed therein so as to communicate with the lumen and intersect a distal tip of the deflecting catheter, the deflecting catheter having a relatively long substantially linear proximal portion and a relatively short substantially linear distal portion, the deflecting catheter being further configured with a pre-formed bend of up to about 85 angular degrees between the proximal and distal portions such that the distal portion is angled relative to the proximal portion so as to have a superior aspect, the channel being formed in the superior aspect and being configured to selectively slidably receive the vascular device from a lateral direction and guide the vascular device axially toward the distal tip.

12. A method of directionally guiding a vascular device, comprising the steps of:
terminating a catheter apparatus in a tapered distal end;
forming a longitudinal channel in the catheter apparatus so as to intersect the tapered distal end;
advancing the catheter apparatus along a first body lumen and into a body cavity;
manipulating the catheter apparatus so as to seat the tapered distal end within a second body lumen;
advancing the catheter apparatus within the second body lumen such that the channel is partially positioned within the second body lumen and partially positioned within the body cavity;
advancing the vascular device along a third body lumen and into the body cavity;
locating the distal tip of the vascular device within the channel;
advancing the vascular device into the second body lumen as guided by the channel; and
retracting the catheter apparatus within the first body lumen to leave the distal tip of the vascular device positioned within the second body lumen.

13. The method of claim 12, comprising the further steps of:
configuring the catheter apparatus with an outer deflecting catheter having a tubular wall defining an interior lumen and terminating at a distal deflecting tip and an inner guiding catheter having a distal guiding tip; forming the channel in the tubular wall so as to communicate with the interior lumen and intersect the deflecting tip; and coaxially and slidably positioning the deflecting catheter about the guiding catheter along at least a portion of the lumen so as to form the catheter apparatus;
seating the guiding tip within the second body lumen;
following the step of advancing the catheter apparatus within the second body lumen, retracting the guiding catheter within the deflecting catheter so as to expose the interior lumen to the body cavity through the channel; and
retracting the deflecting catheter about the guiding catheter so as to effectuate retraction of the catheter apparatus within the first body lumen.

14. The method of claim 13, comprising the further steps of:
configuring the deflecting catheter with a relatively long, substantially linear proximal portion and a relatively short, substantially linear distal portion; configuring the deflecting catheter from a plastic having structural memory; forming a bend of up to about 85 angular degrees between the proximal and distal portions such that the distal portion is angled relative to the proximal portion so as to have a superior aspect, the deflecting catheter being biased to the bend by the structural memory of the plastic so as to have a pre-formed orientation; and forming the channel in the superior aspect;
flexing the distal portion relative to the proximal portion about the bend as dictated by the structure of the first lumen as the catheter apparatus is advanced therein; and
returning the distal portion to the pre-formed orientation when the catheter apparatus is advanced substantially into the body cavity such that the guiding tip is positioned substantially adjacent to the second body lumen.

15. The method of claim 13, comprising the further steps of:
forming the guiding tip with a taper; and
advancing the guiding catheter within the interior lumen such that the guiding tip protrudes beyond the deflecting tip.

16. The method of claim 13, comprising the further steps of:
selecting the body cavity as the right atrium;
selecting the first body lumen as the inferior vena cava;
selecting the second body lumen as the coronary sinus; and
selecting the third body lumen as the superior vena cava.

17. The method of claim 16 wherein the vascular device is selected from the group consisting of a catheter, a guidewire, a balloon catheter, and a pacing electrode catheter.

18. A method of directionally guiding a vascular device, comprising the steps of:
providing an outer deflecting catheter with a tubular wall defining an interior lumen and terminating at a distal deflecting tip;
forming a channel in the tubular wall so as to communicate with the interior lumen and intersect the deflecting tip;
coaxially and slidably positioning a guiding catheter having a tapered distal guiding tip within the deflecting catheter along at least a portion of the lumen so as to form a catheter apparatus;
advancing the guiding and deflecting catheters along a first body lumen and into a body cavity;
manipulating the guiding and deflecting catheters so as to seat the guiding tip within a second body lumen;
advancing the guiding and deflecting catheters within the second body lumen such that the channel is partially positioned within the second body lumen and partially positioned within the body cavity;
retracting the guiding catheter within the deflecting catheter so as to expose the interior lumen to the body cavity through the channel;
advancing the vascular device along a third body lumen and into the body cavity;
locating the distal tip of the vascular device within the channel;
advancing the vascular device into the second body lumen as guided by the channel; and
retracting the deflecting catheter about the guiding catheter within the first body lumen to leave the distal tip of the vascular device positioned within the second body lumen.
